(19) United States
(12) Patent Application Publication
(10) Pub. No.: US 2020/0030440 A1
(43) Pub. Date: Jan. 30, 2020

(54) Financial Method

(71) Applicant: CSL Limited, Parkville (AU)

(72) Inventors: Adriana BAZMORELLI, Parkville (AU); Eugene MARASKOVSKY, Parkville (AU)

(21) Appl. No.: 16/150,631

(22) Filed: Oct. 3, 2018

Related U.S. Application Data
(63) Continuation of application No. 15/282,406, filed on Sep. 30, 2016, now abandoned.
Application No. 15/282,406 is a continuation of application No. 14/447,532, filed on Jul. 30, 2014, now abandoned.

(30) Foreign Application Priority Data
Jul. 31, 2013 (AU) 2013902846

(51) Int. Cl. A61K 39/39; A61K 39/00

(52) U.S. Cl. CPC A61K 39/39; A61K 39/0011; A61K 2039/55511; A61K 2039/55577; A61K 2039/55561; A61K 2039/55572; A61K 2039/55516

(57) ABSTRACT

The present invention provides a composition for raising an immune response against a tumor. The composition comprises at least one tumor antigen, a saponin-based adjuvant, a TLR ligand and a Flt3 ligand.

### RELATED APPLICATIONS

This application is a continuation of U.S. patent application Ser. No. 15/282,406, filed Sep. 30, 2016, pending, which is a continuation of U.S. patent application Ser. No. 14/447,532, filed Jul. 30, 2014, abandoned, which application claims convention priority from Australian Patent application No. 2013902846, filed Jul. 31, 2013, the disclosures of which are incorporated herein by cross reference.

### FIELD OF THE INVENTION

The present invention relates to a compositions for use in cancer immunotherapy and to methods of treating and/or preventing cancer. The compositions comprise one or more tumor antigens in association with a saponin-based adjuvant, a TLR ligand and a Flt3 ligand.

### BACKGROUND OF THE INVENTION

Cancer immunotherapy is the use of the immune system to treat cancer in a patient. The main premise is stimulating the patient's immune system to attack the malignant tumor cells that are responsible for the disease. This can be either through immunization of the patient in which case the patient's own immune system is trained to recognize tumor cells as targets to be destroyed, or through the administration of therapeutic antibodies as drugs, in which case the patient's immune system is recruited to destroy tumor cells by the therapeutic antibodies.

Since the immune system responds to the environmental factors it encounters on the basis of discrimination between self and non-self, many kinds of tumor cells that arise as a result of the onset of cancer are more or less tolerated by the patient's own immune system since the tumor cells are essentially the patient's own cells. Accordingly, whilst the use of a patient's own immune system to target and destroy tumor cells is a well known approach it has often proved difficult to generate a sufficient response in patients.

In addition to the adaptive immune system there is also an innate immune system. Toll-like receptors (TLRs) are a class of proteins that play a key role in the innate immune system. They are single, membrane-spanning, non-catalytic receptors that recognize structurally conserved molecules derived from microbes. Once these microbes have breached physical barriers such as the skin or intestinal tract mucosa, they are recognized by TLRs, which activate immune cell responses.

Toll-like receptors (and other innate immune receptors) are highly specific for the molecules they recognize. These are molecules that are constantly associated with threats (i.e., pathogen or cell stress) and are highly specific to these threats (i.e., cannot be mistaken for self molecules). Pathogen-associated molecules that meet this requirement are usually critical to the pathogen's function and cannot be eliminated or changed through mutation. Well-conserved features in pathogens include bacterial cell-surface lipopolysaccharides (LPS), lipoproteins, lipopeptides, and lipoarabinomannan; proteins such as flagellin from bacterial flagella; double-stranded RNA of viruses; or the unmethylated CpG islands of bacterial and viral DNA; and certain other RNA and DNA.

### SUMMARY OF THE INVENTION

The present inventors have found that by combining a tumor antigen with a particular combination of agents an immune response directed against the tumor which destroys tumor cells can be generated. This combination comprises a saponin-based adjuvant, a TLR ligand and a Flt3 ligand.

Accordingly, in a first aspect the present invention provides a composition, the composition comprising at least one tumor antigen, a saponin-based adjuvant, a TLR ligand and a Flt3 ligand.

In a second aspect the present invention provides a method of treating a tumor in a subject the method comprising administering to the subject a composition comprising at least one tumor antigen associated with the tumor, a saponin-based adjuvant, a TLR ligand and a Flt3 ligand.

In a third aspect the present invention provides a method of protecting a subject against development of a tumor, the method comprising administering to the subject a composition comprising at least one tumor antigen associated with the tumor, a saponin-based adjuvant, a TLR ligand and a Flt3 ligand prior to development of the tumor.

In a fourth aspect the present invention provides a method of inducing an immune response against a tumor in a subject, the method comprising administering to the subject a composition comprising at least one tumor antigen associated with the tumor, a saponin-based adjuvant, a TLR ligand and a Flt3 ligand.

In a fifth aspect the present invention provides the use of a composition comprising at least one tumor antigen, a saponin-based adjuvant, a TLR ligand and a Flt3 ligand in the treatment of a tumor in a subject.

### BRIEF DESCRIPTION OF FIGURES

FIG. 1: Anti-tumor efficacy of combinations of ISCOMATRIX™ adjuvant, Flt3L or Poly IC

FIG. 2: Anti-tumor efficacy of combinations of ISCOMATRIX™ adjuvant+Poly IC and Flt3L, CpG or flagellin

FIG. 3: Anti-tumor efficacy of combinations of ISCOMATRIX™ adjuvant+Poly IC and Flt3L, CpG or flagellin

FIG. 4: Anti-tumor efficacy of combinations of ISCOMATRIX™ adjuvant+Poly IC and Flt3L, CpG or flagellin

FIG. 5: Therapeutic efficacy in B16-OVA melanoma tumor model

FIG. 6: Therapeutic efficacy in TRAMPC1 prostate cancer tumor model

FIG. 7: Effect of vaccination with PAP-ISCOMATRIX™ adjuvant+Poly IC and Flt3L in spontaneous model of prostate cancer

FIG. 8: Extended survival of lymphoma-bearing mice following vaccination with OVA-ISCOMATRIX™ adjuvant, Poly I:C and Flt3-L. One representative result out of two independent experiments is shown. IMX=ISCOMATRIX™ adjuvant.

FIG. 9: Vaccination with OVA-ISCOMATRIX™ adjuvant, Poly I:C and Flt3-L induces antigen-specific lymphoma elimination. The graph shows the number of CD45.2+ CD19+ lymphoma cells in spleens of tumor-bearing mice 13 days after lymphoma inoculation and treated with the indicated treatments. Data are represented as the mean (n=5 mice)±SEM, with each dot representing one mouse. One representative result out of two independent experiments is shown. The p values were calculated using a two-tailed unpaired Student t test. IMX=ISCOMATRIX™ adjuvant.

FIG. 10: Vaccination with OVA-ISCOMATRIX™ adjuvant, Poly I:C and Flt3-L induces reduction in spleen size. The graph shows the weight of spleen of tumor-bearing mice 13 days after lymphoma inoculation and treated with the indicated treatments. Data are represented as the mean (n=5 mice)±SEM, with each dot representing one mouse. One representative result out of two experiments is shown. The p values were calculated using a two-tailed unpaired Student t test. IMX=ISCOMATRIX™ adjuvant.

### DETAILED DESCRIPTION OF THE INVENTION

As discussed above the present invention provides a composition comprising at least one tumor antigen, a saponin-based adjuvant, a TLR ligand and a Flt3 ligand and various uses of this composition. While it is believed that the various elements of this combination are well known to those skilled in the art a brief description of these elements is provided hereunder.

### Saponin-Based Adjuvant

Saponins are steroid or triterpenoid glycosides found in plants, lower marine animals and some bacteria. They contain a steroidal or triterpenoid aglycone to which one or more sugar chains are attached. Steroid saponins can be found in oats, Capsicum peppers, aubergine, tomato seed, alliums, asparagus, yam, fenugreek, Yucca and Ginseng, while triterpenoid saponins have been detected in many legumes such as soybeans, beans, peas, lucerne, etc., and also in alliums, tea, spinach, sugar beet, quinoa, liquorices, sunflower, horse chestnut and Ginseng (Rajput et al. (2007) J Zhejiang Univ Sci B. 8(3): 153-161; Sun et al. (2009) Vaccine 27: 1787-1796).

Saponin-based adjuvants include saponins or saponin derivatives from, for example, Quillaja saponaria, Panax Ginseng Panax notoGinseng, Panax quinquefolium, Platycodon grandiflorum, Polygala senega, Polygala tenuifolia, Quillaja brasiliensis, Astragalus membranaceus and Achyranthes bidentata. For example, a saponin-based adjuvant for use in the vaccines herein can contain Quil A or a Quil A derivative. Quil A is a semi-purified fraction of Quillaja saponins with less toxicity that crude saponin. Quil A is a heterogenous mixture of saponins when analysed by RP-HPLC, containing at least 22 fractions (Kensil et al. (1991) J Immunol 146:431-437). Adjuvant activity is observed in ten of these fractions, including the four most abundant saponins, termed QS7, QS-17, QS-18 and QS-21. QS-21 in particular has been effectively used as an adjuvant. The preparation of QS-21 is well known to those of skill in the art and described, for example, in U.S. Pat. No. 5,057,540. QS-21 can be formulated as an adjuvant with one or more other molecules, such as, for example, 3 De-O-acylated monophosphoryl lipid A (MPL), such as described in International Pat. Pub. Nos. WO 1994000153, WO 1995017210, WO/1998/057660 and WO/2007/068907 (e.g. AS01 and AS02 from GlaxoSmithKline Biologicals). Another saponin-based adjuvant is AS15, which also contains MPL and CpG (GlaxoSmithKline Biologicals, as described in WO 2002/032450). Exemplary saponin-based adjuvants also include semi-synthetic Quillaja saponin analogs, such as those described in U.S. Pat. No. 5,977,081, including the saponin-lipophile conjugate GPI-0100.

Exemplary saponin-based adjuvants also include iscoms (an abbreviation for immuno stimulating complexes) and iscom matrices. This class of adjuvants has been extensively studied and is well known to those of skill in the art (see, e.g. Sjolander et al. (1998) J. Leuk. Biol. 64:713-723; Pearse and Drane (2004) Vaccine 6:4). Iscoms are complexes containing saponin, cholesterol, phospholipid and incorporated protein or proteins (as described, for example, in Sundquist et al. (1988) Vaccine 6:44-48). Iscoms are three dimensional “cage-like” structures, typically about 40 nm in diameter, that form upon detergent removal from mixtures of saponins, detergents and cholesterol. The production and use of iscoms as adjuvants is well known to those of skill in the art and described, for example, in U.S. Pat. Nos. 4,744,983, 4,900,549, 6,352,697 and 6,506,386 and Int. Pat. Pub. No. WO/1987/002250.

Iscom matrices are essentially iscoms without the incorporated protein component. Iscom matrices are usually structurally indistinguishable from iscoms when examined by electron microscopy. Methods for the production and use of iscom matrices, like iscoms, are well known to those skilled in the art and described, for example, in U.S. Pat. Nos. 5,603,958, 5,679,354, 6,352,697, International Pat. Pub. Nos. WO 2002/026255 and WO 2004/004762. Exemplary iscom matrix adjuvants include, but are not limited to, ISCOMATRIX™ adjuvant (CSL Limited), Matrix M™ adjuvant (Isconova, Sweden), Matrix C™ adjuvant (Isconova, Sweden), Matrix Q™ adjuvant (Isconova, Sweden), AbISCO™-100 adjuvant (Isconova, Sweden) and AbISCO™-300 adjuvant (Isconova, Sweden).

As used herein the term “saponin-based adjuvant” refers to an adjuvant that is, contains or includes a saponin or derivative or portion thereof.

### Toll-Like Receptor (TLR) Ligands

There are a range of Toll-like Receptors each of which are specific for particular molecules or classes of molecules. The person skilled in the art is well aware of the ligands which bind particular TLRs, however, information regarding a number of TLRs and their ligands is set out below.

| Receptor | Ligand(s) |
|---|---|
| TLR 1 | multiple triacyl lipopeptides |
| TLR 2 | multiple glycolipids |
| | multiple lipopeptides |
| | multiple lipoproteins |
| | lipoteichoic acid |
| | HSP70 |
| | zymosan (Beta-glucan) |
| | Numerous others |
| TLR 3 | double-stranded RNA, poly I:C |
| TLR 4 | lipopolysaccharide |
| | several heat shock proteins |
| | fibrinogen |
| | heparan sulfate fragments |
| | hyaluronic acid fragments |
| | nickel |
| | Various opioid drugs |
| TLR 5 | flagellin |
| TLR 6 | multiple diacyl lipopeptides |
| TLR 7 | imidazoquinoline |
| | loxoribine (a guanosine analogue) |
| | bropirimine |
| | single-stranded RNA |
| TLR 8 | small synthetic compounds; single-stranded RNA |
| TLR 9 | unmethylated CpG Oligodeoxynucleotide DNA |
| TLR 11 | Profilin |
| TLR 12 | Profilin |
| TLR 13 | bacterial ribosomal RNA sequence |
| | “CGGAAAGACC” |

As used herein the term “TLR ligand” refers to a molecule which is recognized by and binds a Toll-like Receptor.

### F1t3 Ligand

Flt3 ligand recognizes the cytokine receptor CD135. It is an alpha-helical cytokine that promotes the differentiation of multiple hematopoietic cell lineages. Mature human Flt3 ligand consists of a 158 amino acid (aa) extracellular domain (ECD) with a cytokine-like domain and a juxamembrane tether region, a 21 aa transmembrane segment, and a 30 aa cytoplasmic tail. Within the ECD, human Flt3 ligand shares 71% and 65% aa sequence identity with mouse and rat Flt3 ligand, respectively. Human and mouse Flt3 ligand show cross-species activity.

As used herein the term “Flt3 ligand” refers to a molecule which binds CD135. The term includes chimeric molecules which maintain binding to CD135

### Tumor Antigens

Tumor antigens are well known in the art and include products of mutated oncogenes and tumor suppressor genes, products of other mutated genes, overexpressed or aberrantly expressed cellular proteins, tumor antigens produced by oncogenic viruses, oncofetal antigens, altered cell surface glycolipids and glycoproteins and cell type-specific differentiation antigens Examples of tumor antigens include alphafetoprotein (AFP), carcinoembryonic antigen (CEA), CA-125, MUC-1, epithelial tumor antigen (ETA), tyrosinase, Melanoma-associated antigen (MAGE), abnormal products of ras, p53, and glycosphingolipid GD2.

As mentioned above the composition of the present invention comprises at least one tumor antigen, a saponin-based adjuvant, a TLR ligand and a Flt3 ligand. Preferred TLR ligands are TLR3 ligands, TLR4 ligands, TLR5 ligands, TLR 7/8 ligands and TLR9 ligands.

In certain embodiments the saponin-based adjuvant is ISCOMATRIX™ adjuvant, the Flt3 ligand is a chimeric molecule composed of a human Flt3 ligand and a human Fc and the TLR ligand is selected from the group consisting of Poly I:C, CpG, MPL, R848 and flagellin.

Whilst these particular combinations are currently preferred it will be understood that the particular agents specified can be substituted with other agents from the same class, for example another of the many well known saponin-based adjuvants.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

As used in the subject specification, the singular forms “a”, “an” and “the” include plural aspects unless the context clearly dictates otherwise. Thus, for example, reference to “a” includes a single as well as two or more; reference to “an” includes a single as well as two or more; reference to “the” includes a single as well as two or more and so forth.

Having generally described the invention, the same will be more readily understood by reference to the following examples, which are provided by way of illustration and are not intended as limiting.

### EXAMPLES OF THE INVENTION

### Example 1

### Tumor Model B16-OVA

### Methods:

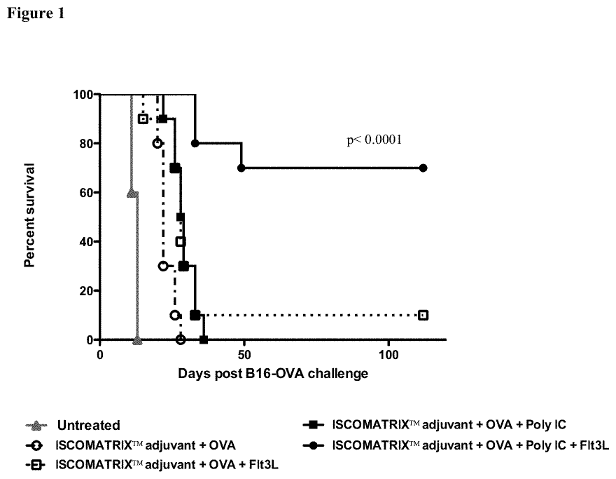

Female C57Bl/6 mice (8-10 weeks old) were dosed with B16OVA cells (5×10⁵ cells) subcutaneously in 100 μl saline in the right flank (anesthetized and shaved with a shaver prior to dosing) with 27 G insulin syringe at day −2. Flt3 ligand (F1t3L, Bioexpress) treatment was also initiated on this day and administered daily for 9 consecutive days. At day 0 (i.e. 2 days after tumor implantation) mice received their first dose of endotoxin free chicken ovalbumin (OVA, Hyglos)+ISCOMATRIX™ adjuvant (+Poly IC). At day 9, mice received second boost dose of OVA+ISCOMATRIX™ adjuvant vaccine. Mice were monitored for tumor growth every 2-3 days. NOTE: OVA (30n)+ISCOMATRIX™ adjuvant (3.8 ISCO™ Units) and Poly IC (5 μg, Invivogen) were delivered as 100 μl dose on day 0 and 7; and Flt3L (10 μg) as a further separate 100 μl dose on days −2 to 7. Mice were culled when tumor reached a size of 10×10 mm. FIG. 1 shows the percent of survival for each group (n=8-10 per group). Data was compared to the group receiving ISCOMATRIX™ adjuvant and OVA and analyzed using Graph Pad Prims version 5. A p value <0.05 was regarded as significant.

### Example 2A

### Tumor Model: Prostate Cancer (TRAMP)

### Methods:

C57Bl/6 male adult mice (8-10 weeks old) were allocated to different experimental groups (n=8-10 per group) as indicated below:

1-Untreated

2-ISCOMATRIX™ adjuvant/PAP

3-ISCOMATRIX™ adjuvant/PAP/Poly IC/F1t3L

4-ISCOMATRIX™ adjuvant/PAP/Poly IC/Flagellin

5-ISCOMATRIX™ adjuvant/PAP/Poly IC/CpG

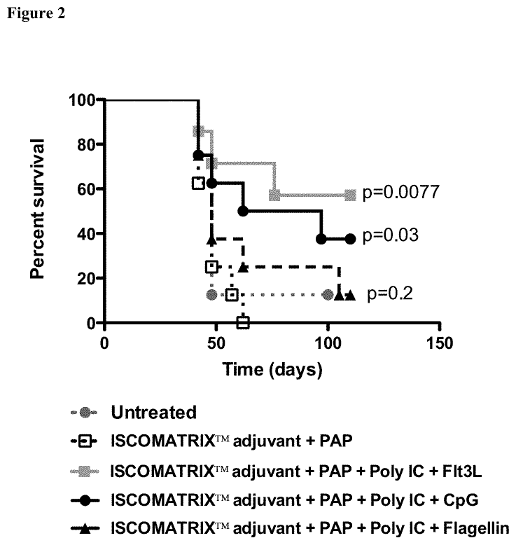

On day 0 mice were anesthetized and injected with 3×10⁶ TRAMP C1 mouse prostate cancer cells in the right flank, subcutaneously (sc). Mice were primed on day 6 and boosted on day 13, with the indicated combination vaccine at the scruff of the neck, sc. Group 3 was inoculated with Flt3L for 9 days starting on day 6, at the scruff of the neck, sc. Mice were culled when tumor reached a size of 10×10 mm. FIG. 2 shows the percent of survival for each group. Data was compared to the group receiving ISCOMATRIX™ adjuvant and PAP and analyzed using Graph Pad Prims version 5. A p value <0.05 was regarded as significant.

Doses were:
ISCOMATRIX™ adjuvant: 3.8 ISCO™ Units.
Poly IC (TLR3 agonist from InVivoGen): 5 μg
Flagellin (TLR5 agonist from Enzo Life Sciences): 200 ng
CpG (1826) (TLR9 agonist from Geneworks): 5 μg
Flt3L-Ig (from BioXpress): 10 μg
PAP (CSL): 300 μg of recombinant mouse prostatic acid phosphatase

### Example 2B

### Tumor Model: Prostate Cancer (TRAMP)

### Methods:

C57Bl/6 male adult mice (6-12 weeks old) were allocated to different experimental groups (n=10 per group) as indicated below:

1-Untreated

2-ISCOMATRIX™ adjuvant/PAP

3-ISCOMATRIX™ adjuvant/PAP/Poly IC/F1t3L

4-ISCOMATRIX™ adjuvant/PAP/Poly IC/Flagellin

5-ISCOMATRIX™ adjuvant/PAP/Poly IC/CpG

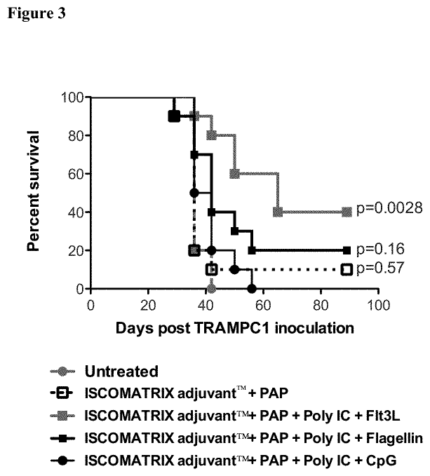

On day 0 mice anesthetized and injected with 3×10⁶ TRAMP C1 mouse prostate cancer cells in the right flank, subcutaneously (sc). Mice were primed on day 2 and boosted on day 9, with the indicated combination vaccine at the scruff of the neck, sc. Group 3 was inoculated with Flt3L for 9 days starting on day 0, at the scruff of the neck, sc. Mice were culled when tumor reached a size of 10×10 mm. FIG. 3 shows the percent of survival for each group. Data was compared to the group receiving ISCOMATRIX™ adjuvant and PAP and analyzed using Graph Pad Prims version 5. A p value <0.05 was regarded as significant.

Doses were:
ISCOMATRIX™ adjuvant: 3.8 ISCO™ Units.
Poly IC (TLR3 agonist from InVivoGen): 5 μg
Flagellin (TLR5 agonist from Enzo Life Sciences): 200 ng
CpG (1826) (TLR9 agonist from Geneworks): 5 μg
Flt3L-Ig (from BioXpress): 10 μg
PAP (CSL): 300 μg of recombinant mouse prostatic acid phosphatase

### Example 2C

### Tumor Model: Prostate Cancer (TRAMP)

### Methods:

C57Bl/6 male adult mice (6-12 weeks old) were allocated to different experimental groups (n=10 per group) as indicated below:

1-Untreated

2-ISCOMATRIX™ adjuvant/PAP

3-ISCOMATRIX™ adjuvant/PAP/Poly IC/F1t3L

4-ISCOMATRIX™ adjuvant/PAP/Poly IC/Flagellin

5-ISCOMATRIX™ adjuvant/PAP/Poly IC/CpG

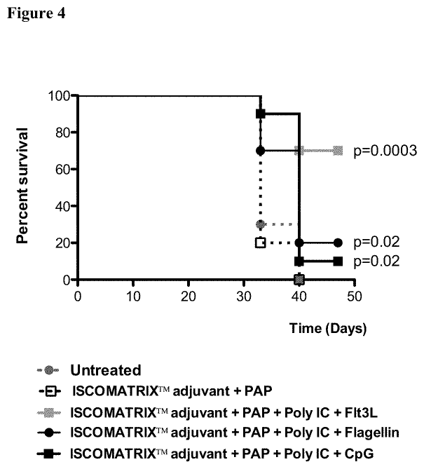

On day 0 mice anesthetized and injected with 3×10⁶ TRAMP C1 prostate cancer cells in the right flank, subcutaneously (sc). Mice were primed on day 2 and boosted on day 9, with the indicated combination vaccine at the scruff of the neck, sc. Group 3 was inoculated with Flt3L for 9 days starting on day 0, at the scruff of the neck, sc. Mice were culled when tumor reached a size of 10×10 mm. FIG. 4 shows the percent of survival for each group. Data was compared to the group receiving ISCOMATRIX™ adjuvant and PAP and analyzed using Graph Pad Prims version 5. A p value <0.05 was regarded as significant.

Doses were:
ISCOMATRIX™ adjuvant: 3.8 ISCO™ Units.
Poly IC (TLR3 agonist from InVivoGen): 5 μg
Flagellin (TLR5 agonist from Enzo Life Sciences): 200 ng
CpG (1826) (TLR9 agonist from Geneworks): 5 μg
Flt3L-Ig (from BioXpress): 10 μg
PAP (CSL): 300 μg of recombinant mouse prostatic acid phosphatase

TABLE 1
Comparison of complete tumor rejection by different combination
vaccines in prostate cancer TRAMP tumor model

| Experiment | Poly(IC) + | Poly(IC) + | |
|---|---|---|---|
| Number | Flt3L | Flagellin | Poly(IC) + CpG |
| Example 2A | 62 | 35 | 40 |
| Example 2B | 40 | 20 | 0 |
| Example 2C | 30 | 0 | 0 |
| No. tumor free | 12/28 | 5/28 | 4/28 |
| mice/Total number | | | |
| (*) | | | |

(*) Data correspond to experiments 2A, 2B and 2C combined

### Example 3

### Therapeutic Efficacy of Vaccines Comprising ISCOMATRIX® Adjuvant, Flt3L and TLR Agonists in a Mouse Model for Melanoma or Prostate Cancer.

### Methods

C57Bl/6 adult female mice were injected with 5×10⁵ B16-OVA and C57Bl/6 adult male mice were injected with 3×10⁶ TRAMPC1 tumor cells at the right flank, subcutaneously (sc). Chicken ovalbumin at 30 μg (OVA) or prostatic acid phosphatase at 300 μg (PAP) were used as tumor antigens for melanoma and prostate cancer tumor models, respectively. On days 2 and 9 mice were immunized with the indicated vaccines and Flt3L was administered for nine days starting on day of tumor inoculation. Vaccines and Flt3L were injected at the scruff of the neck sc.

Experimental groups (n=10)
ISCOMATRIX® adjuvant+tumor antigen
ISCOMATRIX® adjuvant+tumor antigen+Poly IC+Flt3L
ISCOMATRIX® adjuvant+tumor antigen+CpG+Flt3L
ISCOMATRIX® adjuvant+tumor antigen+R848+Flt3L
ISCOMATRIX® adjuvant+tumor antigen+MPL+Flt3L
ISCOMATRIX® adjuvant+tumor antigen+Flagellin+Flt3L

Doses for each vaccine component were:

| Vaccine Component | Dose |
|---|---|
| ISCOMATRIX ® adjuvant | 3.8 ISCO ™ Units |
| Poly IC, CpG, MPL | 5 μg |
| R848 | 10 μg |
| Flagellin | 200 ng |
| F1t3L | 10 μg |

Flagellin was purchased from Enzo Life Sciences and all other TLR agonists from InVivoGen. Flt3L was purchased from Bio Xpress

Vaccine efficacy was assessed by tumor growth, percentage of tumor free mice and percent of survival. Percent of survival data was analyzed using Long-rank (Mantel-Cox) test. Differences were regarded as significant if p<0.05.

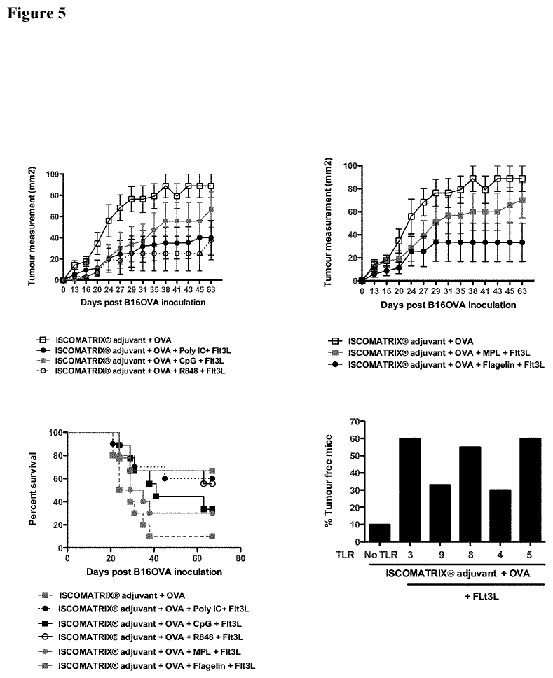

The results obtained in the B16-OVA melanoma experiments are shown in FIG. 5. Statistical analyses of percent of survival at the end of experiment was as follows:

| | P value (comparison with |
|---|---|
| TLR agonist in | ISCOMATRIX ® |
| the vaccine | adjuvant + OVA group) |
| Poly IC (TLR3) | 0.01 |
| CpG (TLR9) | 0.05 |
| R848 (TLR7/8) | 0.02 |
| MPL (TLR4) | 0.17 |
| Flagelin (TLR5) | 0.02 |

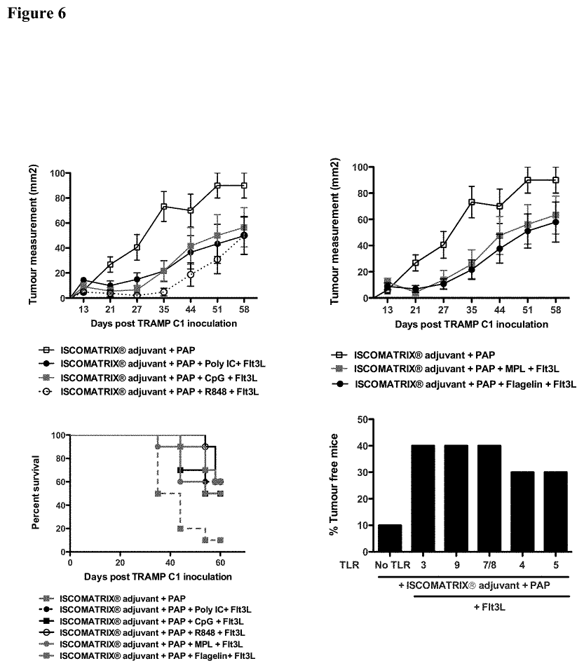

The results obtained in the TRAMPC1 prostate cancer experiments are shown in FIG. 6. Statistical analyses of percent of survival at the end of the experiment was as follows:

| TLR agonist in | P value (comparison with |
|---|---|
| the vaccine | ISCOMATRIX ® adjuvant + PAP group) |
| Poly IC (TLR3) | 0.005 |
| CpG (TLR9) | 0.01 |
| R848 (TLR7/8) | 0.00006 |
| MPL (TLR4) | 0.02 |
| Flagelin (TLR5) | 0.002 |

### Example 6

Vaccination with PAP-ISCOMATRIX™ Adjuvant-Poly LC and Flt3L Treatment Induces Tumor Control in a Spontaneous Model of Prostate Cancer

### Methods

Transgenic TRAMP (TRansgenic Adenocarcinoma of the Mouse Prostate) mice start to develop prostate cancer spontaneously at 12 weeks of age, following puberty. TRAMP mice were vaccinated with PAP-ISCOMATRIX™ adjuvant-Poly I:C twice one week apart, subcutaneously at the scruff of the neck. First vaccination was performed between weeks 6-8. Mice were also injected for 9 consecutive days with Flt3L, sc at the scruff of the neck. First dose of Flt3L was performed two days prior to priming with the vaccine.

Mice were killed on week 21-24 and the weight of prostate and vesicles was determined. Untreated non-transgenic littermates were used as negative controls.

### Groups

1. non-transgenic littermates mice (n=26)
2. TRAMP mice untreated (n=29)
3. TRAMP mice vaccinated (n=22)

### Results

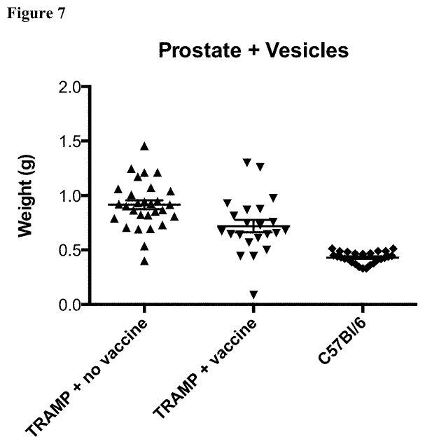

The result are shown graphically in FIG. 7. Significant lower prostate (p=0.002) and vesicle weight was observed in mice treated with Flt3L and vaccine compared with unvaccinated TRAMP mice. This result suggests that the vaccine induces significant control of tumor growth in the prostate. Data pooled from 10 independent experiments.

### Example 7

Therapeutic Vaccination with OVA-ISCOMATRIX™ Adjuvant, Poly I:C and Flt3-L Treatment Induces Lymphoma Control in a Mouse Model of Blood Cancer.

### Materials & Methods

The mouse Eμ-myc B-cell lymphoma is a well-established model of human Burkitt's lymphoma. Eμ-myc cells that express the reporter protein GFP and the tumor model antigen Ovalbumin (Eμ-myc-GFP-OVA) were used to assess the efficacy of anti-lymphoma vaccines.

Wild type (CD45.1+) host mice were injected intravenously with 1,000 (CD45.2+) Eμ-myc-GFP-OVA lymphoma cells. Two days later, lymphoma-bearing mice were vaccinated with OVA-ISCOMATRIX™ Adjuvant and Poly I:C, twice one week apart, subcutaneously at the scruff of the neck. In addition, mice were also injected for 9 consecutive days with Flt3-L, subcutaneously at the scruff of the neck. This Flt3-L treatment started at the time of the lymphoma inoculation. Tumor-bearing mice were monitored daily for signs of illness characterized by ruffled fur, hunched back and/or inactivity. When mice showed signs of advanced ill-health, they were euthanized and their spleen was harvested for analysis of tumor burden.

### Groups:

1. Untreated (n=5)
2. OVA-ISCOMATRIX™ adjuvant (n=5)
3. OVA-ISCOMATRIX™ adjuvant+Poly I:C+Flt3-L (n=5)

### Results

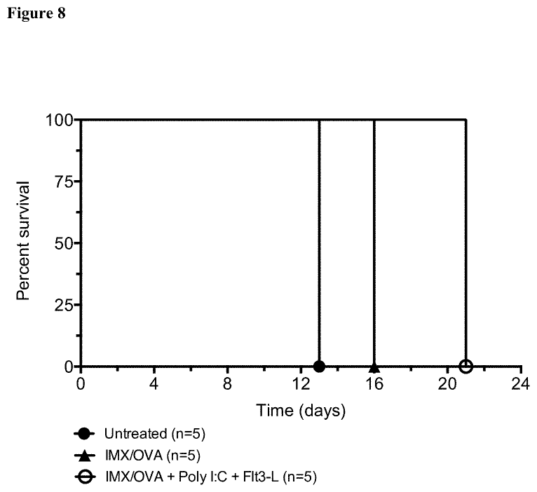

Lymphoma-bearing mice developed advanced illness on day 13 post-lymphoma inoculation if they were untreated with a vaccine (FIG. 8). In contrast, mice vaccinated with OVA-ISCOMATRIX™ adjuvant, Poly I:C and Flt3-L had their lifespan 60% extended (FIG. 8).

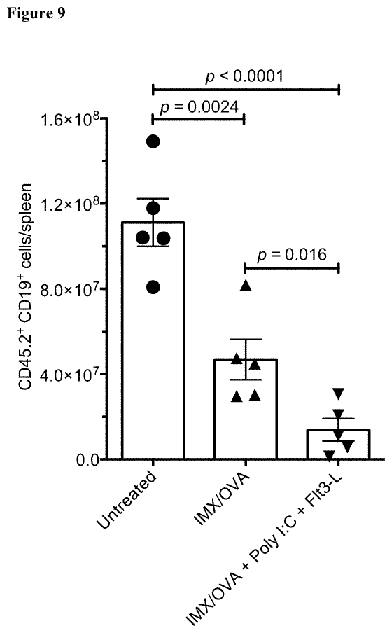

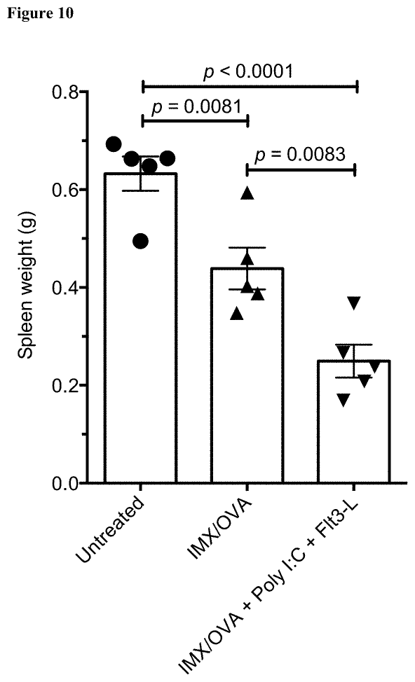

To confirm tumor elimination in mice vaccinated with OVA-ISCOMATRIX™ adjuvant, Poly I:C and Flt3-L, spleens of mice were harvested and analyzed for tumor burden 13 days following lymphoma inoculation. At this time, untreated lymphoma-bearing mice had advanced illness and had to be euthanized (FIG. 8). Analysis of the spleens from mice treated with OVA-ISCOMATRIX™, Poly I:C and Flt3-L showed a significant reduction in the number of lymphoma cells compared to control untreated mice or mice vaccinated with OVA-ISCOMATRIX™ adjuvant alone (FIG. 9). This reduction in tumor burden correlated with a significant reduction in spleen size (FIG. 10).

1-16. (canceled)

17. A method for providing paid selective personalized health care services to at least one individual in a population of individuals, comprising: (a) establishing a record of information representative of the status of an individual in the population; (b) analyzing said record according to one or more defined algorithms; (c) generating a recommendation to the at least one individual based upon said analysis, and (d) charging the at least one individual a fee for the recommendation, wherein if the status indicates that the at least one individual has a tumor or is at risk of developing a tumor, the recommendation that is generated recommends administration of a composition to the at least one individual, wherein the composition comprises at least one tumor antigen, a saponin-based adjuvant, a TLR ligand and a Flt3 ligand.

18. The method of claim 1 in which the saponin-based adjuvant is an ISCOMATRIX adjuvant.

19. The method of claim 1 in which the Flt3 ligand is a chimera of human Flt3 ligand and human Fc.

20. The method of claim 1 in which the TLR ligand is a TLR 3 ligand.

21. The method of claim 4 in which the TLR 3 ligand is Poly IC.

22. The method of claim 1 in which the TLR ligand is a TLR 4 ligand.

23. The method of claim 6 in which the TLR 4 ligand is monophosphoryl lipid A (MPL).

24. The method of claim 1 in which the TLR ligand is a TLR 5 ligand.

25. The method of claim 8 in which the TLR 5 ligand is Flagellin.

26. The method of claim 1 in which the TLR ligand is a TLR 7/8 ligand.

27. The method of claim 10 in which the TLR 7/8 ligand is imidazoquinoline (R848).

28. The method of claim 1 in which the TLR ligand is a TLR 9 ligand.

29. The method of claim 12 in which the TLR 9 ligand is CpG.


SEQUENCE LISTING (1 sequence)

SEQ ID NO: 1 - RNA, 10 nt, Artificial Sequence
  Bacterial ribosomal RNA sequence
cggaaagacc